(19) United States
(12) Patent Application Publication
(10) Pub. No.: US 2022/0071803 A1
(43) Pub. Date: Mar. 10, 2022

(54) System and Method for Vaginal Remodeling

(71) Applicants: Bandula Wijay, Friendship, TX (US); Nandhika Wijay, Friendship, TX (US); Constanza Wijay, Friendship, TX (US)

(72) Inventors: Bandula Wijay, Friendship, TX (US); Nandhika Wijay, Friendship, TX (US); Constanza Wijay, Friendship, TX (US)

(21) Appl. No.: 17/016,322

(22) Filed: Sep. 9, 2020

(51) Int. Cl. A61F 7/12; A61H 9/00; A61H 19/00; A61N 1/05; A61N 1/36

(52) U.S. Cl. CPC A61F 7/123; A61H 9/0021; A61F 2007/005; A61N 1/0524; A61N 1/36007; A61H 19/40

(57) ABSTRACT

A system and method for vaginal remodeling and treating gynecological disorders. A vaginal remodeling system comprising a flexible, inflatable, vaginal balloon capable of transferring heat and mechanical stress in a controlled manner to restore vaginal function. The proposed method to reactivate the cells in the vaginal tissue using thermal energy and mechanical energy using balloon dilatation and/or pulsing to promote the rejuvenation of the vaginal tissue will be accomplished by the system described in this disclosure.

### BACKGROUND

Vaginal anatomy and workings are complex. The vaginal wall tissue consists of a fibromuscular tube that connects the uterus with the external female genitals. The vagina has three layers: the internal mucosal layer, the middle muscularis layer, and the external adventitial layer. The internal mucosa layer consists of epithelial cells that undergo hormone related cyclical changes. The middle muscularis layer allows vaginal tube to contract and relax. The adventitial is the portion of the vaginal wall that provides structural support and comprises collagen and elastin. Nerves form the uterovaginal plexus and other sources provide complex vascular support. In a sexually stimulated state, potassium ions and sodium ions concentrate in the internal mucosal layer, and the potassium ion concentration is typically higher than the sodium ion concentration. Capillaries transport sodium ions to the plasma in the in the underlying tissue bed. With poor circulation, sodium ion transport is impaired.

Vaginal atrophy can occur due to decreased production of estrogen, child birth, menopause, sexual inactivity, and/or ageing. The vaginal tube structure thins, and the vaginal tube structure can become relaxed, e.g., Vaginal Relaxation Syndrome (VRS), tightened, e.g., Atrophic Vaginitis. Either of these conditions can cause physical and emotional trauma to a woman. Both VRS and Atrophic Vaginitis can also cause urinary incontinence.

Sometimes Atrophic Vaginitis can cause the vaginal tube structure to become so tight that a slim structure, such as a finger, cannot be introduced to the vaginal canal.

The etiology for any vaginal problems is complex and difficult to pinpoint; hence the lack of one unique solution. The exact mechanisms and reasons for these outcomes are well addressed in the literature and root causes of cell behaviors are so intermingled, no one cause can be identified and therefore addressed. Multiple inventions to solve these problems are in the patent literature as well as other non-patented devices have been proposed and marketed. These include but not limited to various devices projecting light using light emitting diodes (LED) to thermal means, low energy laser light, radio frequency energy.

The general philosophy in all these treatments is to reactivate the cells in the vaginal mucosa tissue, activate collagen and elastin function by remodeling the collagen and elastin within the connective tissue. The heat and mechanical energy enhance the collagen remodeling inducing an inflammatory response and fibrotic activity within the vaginal tissue and hence some tightening of the vaginal muscle.

Multiple inventors have patented devices and methods to alleviate and resolve vaginal atrophy. Tettamani et.al. in U.S. Pat. No. 8,709,057 describes a method of using non-ablative laser energy in the 1.9-11 micrometer wavelength pulses to deliver energy of 1.9-2.5 Joules per square centimeter. Takacs et.al. in U.S. Pat. No. 9,375,450 uses Zinc Sulfate to promote smooth muscle cell proliferation as means to revitalize the vaginal wall. Courtion et.al in U.S. Pat. No. 9,949,899 uses light from diodes and simultaneous vibration to rejuvenate the vaginal wall. Courtion et.al in U.S. Pat. No. 10,179, 085 uses low level laser light using laser diodes to along with simultaneous vibration to rejuvenate the vaginal wall atrophy.

While these patented methods using energy in the form of heat, light, and mechanical vibration seem logical for rejuvenating cells in the human body, these concepts have not had significant success providing a solution to vaginal atrophy or vaginal relaxation syndrome. Further, several commercially available devices such as Monolisatouch®, Vieve®, and Thermiva® have been used by doctors to resolve vaginal issues; however, success has been limited.

There remains a need for a gynecological device that successfully treats vaginal atrophy and vaginal relation syndrome.

### SUMMARY

The present invention proposes a solution to the complex issues associated with treating vaginal atrophy and vaginal relaxation syndrome (VRS).

In at least one embodiment of the present disclosure, a dilatation balloon capable of mechanically inducing stress in the vaginal wall while providing a pulsating motion to stretch and relax the vaginal wall. The frequency and the degree of pulsations can be adjusted to the desired level. As an example, a pulse cycle can comprise one to ten pulses per minute as a therapy for vaginal atrophy and VRS.

In at least one embodiment of the present disclosure, the balloon can be uniformly heated by adding a heated fluid to the interior of the balloon. The heated fluid may be saline, and the heated fluid may be pumped into the balloon by a pump.

In at least one embodiment of the present disclosure, the fluid may be heated by low voltage heating element within the balloon, to provide heat energy to stimulate cell function, remodeling of collagen, and facilitate blood flow in the capillary bed and other regions of the vaginal tube structure.

In at least one embodiment of the present disclosure, a balloon is introduced into the vagina in a folded state, wherein the balloon may comprise a very low profile and can be relatively non-distensible or semi-compliant. The balloon can be deflated and empty at the time it is inserted into the vagina. Folding the balloon prior to introducing it into the vagina by inserting it therein can reduce the discomfort and pain for an atrophied vagina.

In at least one embodiment of the disclosure, a lidocaine cream or other numbing or anesthetic cream or spray may be applied to the inside and outside of the vagina and on the balloon before a balloon is inserted to reduce pain.

In at least one embodiment of the present disclosure, a catheter may comprise a shaft having a proximal end and a distal end. The distal end of the balloon is attached to the distal end of the shaft and the proximal end is attached towards to proximal end of the catheter shaft. The catheter may comprise a diameter ranging from 10 mm to 40 mm, 15 mm to 35 mm, 18 mm to 33 mm, 10 mm to 20 mm or any diameter in any of the ranges, and be made from an appropriate material such as biaxially oriented Nylon®, Pebax®, polyurethane, or silicone. A balloon catheter may comprise an inflation lumen as well as a through lumen to advance a wire if necessary. However, a through lumen is not essential for the functionality of the afore mentioned device.

In at least one embodiment of the present disclosure, the balloon can be inflated with a commercially available inflation syringe using water or saline.

In at least one embodiment of the present disclosure, the balloon can be inflated with an inflation pump which is capable of providing a continuous stream of heated inflation fluids.

In at least one embodiment of the present disclosure, the inflation pump can control the inflation and deflation of the balloon according to a preprogrammed cycle of inflation and deflation while maintaining the temperature of the inflation fluid. The cycle of inflation and deflation can create a pulsing of the balloon while it is located inside a vagina.

In at least one embodiment of the present disclosure, a vaginal remodeling system comprising at least a flexible balloon catheter designed for insertion into any size vagina is described.

In at least one embodiment of the present disclosure, a vaginal remodeling system can comprise temperature regulation.

In at least one embodiment of the present disclosure, a vaginal remodeling system can provide mechanical stress to the interior of a vagina.

In at least one embodiment of the present disclosure, a vaginal remodeling system can comprise a means for generating pulsing mechanical stimulation inducing stress of a vaginal wall.

In at least one embodiment of the present disclosure, a vaginal remodeling device can comprise a means for generating a variably timed mechanical pulsing.

In at least one embodiment of the present disclosure, a vaginal remodeling device can provide electrical stimulation to an inside of a vagina to cause muscular contraction of the vaginal wall.

In at least one embodiment of the present disclosure, a vaginal remodeling device can provide pulsing electrical stimulation to an inside of a vagina.

In at least one embodiment of the present disclosure, a vaginal remodeling system can provide a variably timed electrical pulsing for stimulating and contracting the vaginal tube structure.

Those skilled in the art will further appreciate the above-mentioned advantages and superior features of the invention together with other important aspects thereof up reading the detailed description which follows in conjunction with the drawings.

### BRIEF DESCRIPTION OF THE DRAWINGS

FIG. 1 illustrates a top view of at least one embodiment of a vaginal remodeling system in a vagina.

FIG. 2 illustrates a top view of at least one embodiment of a vaginal remodeling system in the vagina, consisting of the balloon catheter, pacing electrodes and the pumping means, with fitting 245 shown.

FIG. 3 illustrates a top view of at least one embodiment of a vaginal remodeling system comprising a balloon catheter, a balloon inflation means, and pump means.

FIG. 4 is an illustration of at least one embodiment of a vaginal remodeling system comprising a balloon catheter, along with an inflation syringe and a pulsating means having fluid heating capability, which are all parts of at least one embodiment of a vaginal remodeling system.

FIG. 5 is an illustration of at least one embodiment of a pump means to control, inflate, and/or pulse a balloon catheter using a supply of heated fluids during the inflation-deflation of the balloon to provide an effective circulation of the heated fluid through the vaginal remodeling system.

FIG. 6 is an illustration of at least one embodiment of a potential display for the device control module.

FIG. 7 is an illustration of at least one embodiment of a connector and locking mechanism for preventing the reuse of the vaginal remodeling system by the user.

### DESCRIPTION OF THE PREFERRED EMBODIMENTS

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor it is to be construed, to limit the boundaries of the descriptions but rather as defined by the claims and equivalents thereof. Therefore, embodiments not specifically addressed herein, such as adaptations, variations, modifications, and equivalent arrangements, should be and are considered to be implicitly disclosed by the illustrative embodiments and claims set forth herein and therefore fall within the scope of the present invention.

Further, it should be understood that, although steps of various claimed methods may be shown and described as being in a sequence or temporal order, the steps of any such method are not limited to being carried out in any particular sequence or order, absent an indication otherwise. That is, the claimed method steps are considered capable of being carried out in any sequential combination or permutation order while still falling within the scope of the present invention.

Additionally, it is important to note that each term used herein refers to that which a person skilled in the relevant art would understand such term to mean, based on the contextual use of such term herein. To the extent that the meaning of a term used herein, as understood by the person skilled in the relevant art based on the contextual use of such term, differs in any way from any particular dictionary definition of such term, it is intended that the meaning of the term as understood by the person skilled in the relevant art should prevail.

Furthermore, a person skilled in the art of reading claimed inventions should understand that “a” and “an” each generally denotes “at least one,” but does not exclude a plurality unless the contextual use dictates otherwise. Also, the term “or” denotes “at least one of the items,” but does not exclude a plurality of items of the list.

In the description which follows, like parts are marked throughout the specification and drawings with the same reference numerals, respectively. The drawing figures may not necessarily be to scale and certain features may be shown in somewhat schematic form in the interest of clarity and conciseness.

Most devices described in the prior art are rigid or semi rigid devices with a solid body and a solid surface. While these can be introduced into the vagina that is highly extended, it cannot be easily or properly introduced into a very tight vagina. Further, while some devices are used in combination therapy with oral and/or topical hormones, current solutions fail to address the under lying issue of activating the collagen and elastin tissues. Vaginal rejuvenation by neocollagenasis is a basic requirement in remodeling the collagen in the vaginal tissue, along with creating an inflammatory response resulting fibroblast response for the remodeling of the cellular and extra cellular matrix.

While only using energy sources may help some, combining mechanical dilatation of the vaginal lumen with heat can effectively cause the necessary tissue reaction. In addition, either by itself or along with heat and dilatation, the vaginal tube can be physically manipulated to create mechanical disruption to the vaginal tissue. Using an external force to dilate the vaginal tissue promotes the breakdown of the old tissue layers in the vaginal wall and creation of new tissue. This action, helped by providing heat energy which in turn adds to this effort by facilitating capillary blood flow in the vaginal tube promoting healthy tissue growth and restoring cellular function.

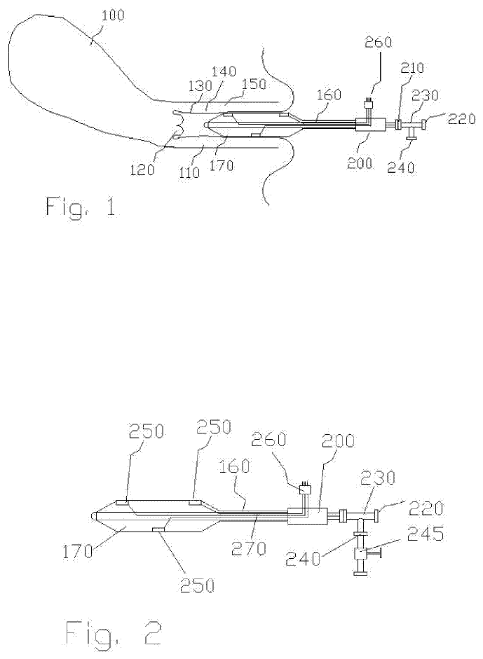

Referring to FIG. 1, there is illustrated at least one embodiment of a vaginal remodeling system 100. The vaginal remodeling system 100 is shown positioned within a vaginal tube 110. A uterus 100 is connected to the vaginal tube 110 with a cervix 120 therebetween and defining the distal end of the vaginal tube 110 and proximal end of the uterus 100. The vaginal tube 100 comprises an internal mucosal layer 130, a middle muscularis layer 140, and an external adventitial layer 150 (collectively an interior portion of the vaginal tube 100).

A vaginal dilatation balloon catheter 160 having a balloon 170 can be made from a relatively non-distensible material, such as Nylon® L2101F or similar material. The balloon 170 can be made by blowing air to into an extruded Nylon® tube placed within a heated mold of desired size and shape, while stretching the tube. Thus, causing a thin wall Nylon® balloon with biaxially oriented molecular structure in its wall imparting high tensile strength both in circumferential and lengthwise direction. The balloon made in this manner can withstand high inflation pressures and temperatures that are above the normal glass transition temperature of the particular Nylon material. Alternatively, materials such as PEBAX®, polyethylene terephthalate (PET), or crosslinked polyethylene may also be used as a balloon material for the above purpose, among other suitable materials.

The balloon catheter 160 comprises a shaft 165 that can be affixed to the balloon 170 using a “Y” or “T” connection at its proximal hub 200 that will provide at least one lumen connection. A first lumen connection 210 and/or a second lumen 220 are shown for exemplary purposes. The first lumen connection 210 and/or the second lumen connection 220 can be used for inflating and/or deflating the balloon 170 with an appropriate fluid heated to the appropriate temperature. The proximal hub 200 can also be connected to a “T” connector 230, so that the balloon 170 can be initially inflated by connecting the port 240 to an inflation syringe 235 equipped with a pressure gage.

The balloon 170 can be inserted into the vaginal tube 110 when it is a deflated state. The balloon 170 can be folded to reduce its size when inserting it into the vaginal tube 110. Once the balloon 170 is positioned into a desired place inside the vaginal tube 110, it can be inflated with a warm or heated fluid, heated to 37-60 degree Centigrade, such as water or saline, to the predefined diameter. An inflation syringe (see FIG. 3) may be employed to move the fluid contained in the inflation syringe or in a fluid container that is in fluid communication with the inflation syringe or piston and cylinder arrangement into the balloon 170 through the balloon catheter 160.

Depending on the anatomy and severity of the patient's gynecological disorder, the operator can choose an appropriate size of balloon 170. The size of the balloon 170 can range from 1 cm diameter to 4 cm in diameter, or any incremental measure therebetween, for the procedure. The balloon 170 may be partially inflated, and may be inflated at a very slow rate, depending on the pain level the patient can tolerate. With time, the patient may be able to tolerate the full desired inflation. Once inflated, the balloon 170 can be left in place for a period of time, as an example, ranging from 1-15 minutes, while maintaining the temperature of the fluid inside the balloon 170 by replenishing the inflation fluid, with fluid at the desired temperature.

The balloon 170 may be deflated and re-inflated (pulsated) at a regular interval to stretch and release the stretch of the vaginal tube 110 to stress and stimulate the tissue and cells of the vaginal tube 110. The vaginal remodeling system 10 has the capability to use the inflation, pulsating, heating functions together or independently depending on the patient's ability to withstand pain and any discomfort.

The vaginal remodeling system 10 can also treat VRS. Treating VRS requires additional exercise of the vaginal muscle, similar to Kegel exercises. Electrodes may be placed on the outer surface of the balloon 170 to provide electrical stimulation pulses to cause muscle contraction in the vaginal tube 110.

The electrical stimulation pulses may be rhythmed, as desired, to create stress on the tissue and cells of the vaginal tube 110. The electrical stimulation pulses will cause the vaginal tube 110 to contract and relax for a period of time during the treatment. The balloon 170 can be utilized in conjunction with the electrical stimulation pulses and inflated with the heated fluid, at the same time, or sequentially to provide additional stimulation to the vaginal tube 110. The balloon 170 inflated with heated fluid can provide heat energy to the vaginal tube 110 or it can be accompanied with mechanical pulsation as previously described. All these different actions can be performed at the same time or separately. The best mode can be decided by the attending physician based on the severity of the vaginal atrophy and the VRS.

Turning to FIG. 2, the balloon catheter 160 comprising the balloon 170 attached distally, and multiple pacing electrodes 250 affixed to the wall of the balloon 170 can be seen. The multiple pacing electrodes 250 can be placed at predetermined locations on the wall of the balloon 170 to facilitate the desired pacing of the contractions of the vaginal tube 170. The multiple pacing electrodes 250 can be connected to an electrical connector 260 using thin electrical conductor means 270, which can be printed onto the wall of the balloon 170 and the adjoining catheter tube wall, or can be a fine silver or copper wire extending from the multiple pacing electrodes 250 to the connector 260.

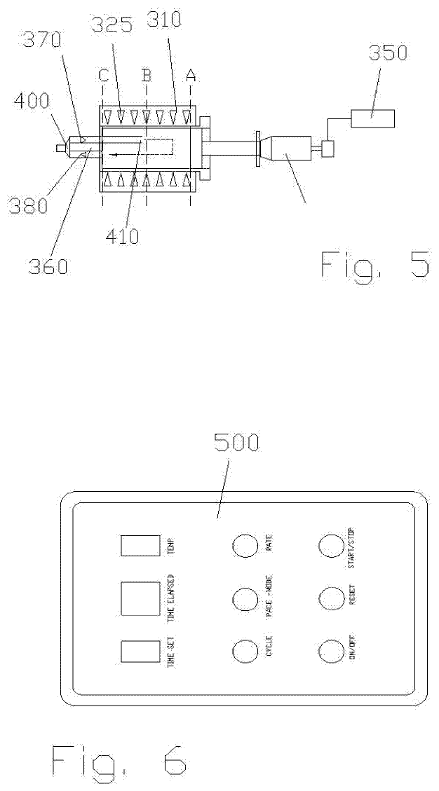

The connector 260 can be connected to a control unit 350 (see FIG. 5), comprising an electronic module that can appropriately pace the electrical stimulation pulses sent to the vaginal tube 110 to cause contractions in the vaginal tube muscularity.

Turning to FIG. 3, inflation fluid can be introduced at the port 240 of the “T” connector 230. Once the balloon 170 is inflated to the appropriate pressure using an inflation syringe 235, e.g., to 4-6 atm. pressure or any pressure between 0 atm and 5 atm, the port 240 can be closed using a stopcock 245. The pressure can be cycled from no pressure (0 atm.) to its highest rated pressure (4-6 atm.) using a pumping means 300 whereby the heated inflation fluid, e.g., heated between 40 and 70 degrees Centigrade, and at pressure between 4-6 atm, is circulated. The pressure cycling can also be from any fraction of the rated maximum pressure to the rated pressure.

Pressure cycling between 25-100%, 50-100%, 75-100% would be adequate to treat the gynecological disorder (vaginal atrophy or VSR), though the pressure cycle levels would be determined based on the patient's pain level and the stage of the treatment process. As an example treatment method, though this disclosure is not to be limited to this example, the first treatments can be from 75-100% and as the patient's vaginal disorder improves, these can be increased gradually. Any other combination of therapy can be decided by the attending physician.

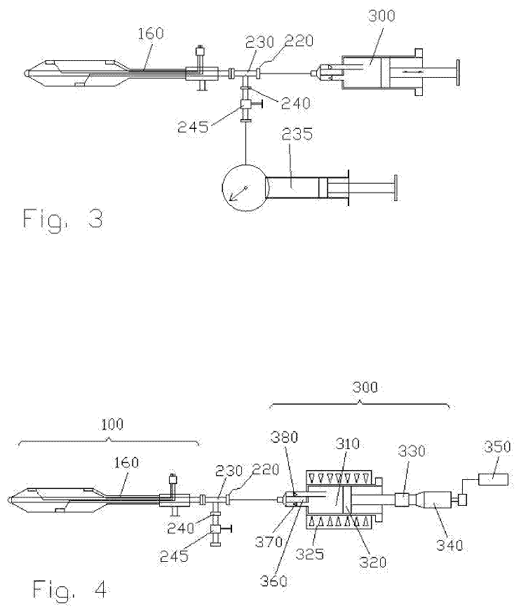

Turning to FIG. 4, at least one embodiment of a vaginal remodeling system 10 can be seen. The vaginal remodeling system 10 can comprise a pump 300, valves 370 and 380, crosshead 330, motor 340, and programmable electronic control system 350 associated with the fluid pumping system 300. The vaginal remodeling system 10 can comprise a pump cylinder 310 made from a material such as stainless steel or high temperature plastics if the pump cylinder is to be disposed, a piston 320 made from Nylon®, Teflon® or PEEK for elevated temperature functionality having a sealing “0” ring, a crosshead 330 which moves the piston back and forth in a linear motion and can be attached to a motor 340 capable of driving the crosshead 330. The motor 340 can rotate clockwise and counter clockwise, enabling the reverse of the direction of motion of the crosshead 330 and can be connected to a programmable electronic control system 350.

The crosshead 330, motor 240 and programmable electronic controller system 250 can be programmed to engage the piston 320 and move the piston 320 back and forth. The piston 320 may be actuated and either move through a complete cycle, whereby all the fluid in the cylinder is pumped in each stroke, or a partial cycle, where only a portion of the fluid is pumped in each stroke. The piston 320 provides a pumping action as well as pulsatile action when the fluid is pumped through balloon catheter 160 into the balloon 170. The programmable electronic control system 350 can be programmed to set a flow rate at which the pump piston 320 is actuated.

The programmable electronic control system 350 can be used to program the pump 300 such that the rate of inflation is controlled depending on the pain tolerance level of the patient. The programmable electronic control system 350 can also be programmed to control the pump 300 such that the rate of inflation is controlled depending on the extent that the balloon 170 is inflated (i.e. to the percentage of the diameter of the balloon), thereby providing a discretion for the doctor to determine the extent to which the vaginal tube 110 is stretched and to what degree and how fast the vaginal tube 110 is stretched. In addition, the temperature is also controlled to the desired thermal treatment level by the doctor.

In at least one embodiment of the present disclosure, the pump cylinder 310 can be heated with heating means 325 to a desired temperature, e.g., 40-70 degree Centigrade or any degree within 40-70 degree Centigrade, is maintained using the electronic temperature controls available within the control unit 350.

The pump cylinder 310 can be connected to a manifold 360, which comprises two valves 370 and 380. The valve 370 opens for the fluid to flow out of the pump (in out-flow mode) and the valve 380, which opens when the fluid leaves the pump in the in-flow mode.

The pump cylinder 310, the piston 320, and the two one-way valves 370 and 380 can work together to create a pumping action for the pump 300 due to the workings of the crosshead 330, motor 340, and the programmable electronic controller system 350.

Turning to FIG. 5, at least one embodiment of a pumping unit 385 is illustrated. The pumping unit 385 can be battery operated or operated by connecting to a 110V wall power plug. The pump cylinder 310 comprises at least two sections. Section A-B can be the portion in which the piston 320 moves in a reciprocal manner. Section B-C can be the portion comprising a fluid. Heating elements 325 heat the fluid in Section B-C before the fluid is pumped into the balloon catheter 160 (see FIG. 4). The fluid returning from the balloon catheter 160 (see FIG. 4) can enter the pump cylinder 310 through the manifold 400 and move through the one-way valve 370 at a position close to the reference dashed line “C”. The retuning fluid has lost heat and transferred the heat to the vaginal tube 110 (see FIG. 1) and therefore has a lower temperature than when it was pumped into the balloon catheter 160 (see FIG. 4). The fluid entering the pump is placed between the section “B” and “C” in order to heat them before pumping out again. The outlet tube 410 can be positioned close to the line “B”, so that when the piston 320 is in the pumping mode stroke, the fluid that has been heated is forced out of the pump thereby providing a circulating means within the pump cylinder 300. During this stroke, the one-way valve 380 is positioned in the opposite direction and therefor prevents any fluid from the pump cylinder 310 from entering the outlet port 400 and allows the fluid to be heated by the heating elements 325.

When the piston 320 is in the pumping mode, the one-way valve 370 remains open and the one-way valve 380 closes, and as such the fluid which is in the space between the lines “B” and “C”, which has been heated by the heater 325 is pumped out of the pump cylinder 310, into the balloon catheter 160, and into the balloon 170. By this arrangement, the fluid travels in a one-way manner and allows cold or cooled fluid to enter the pump unit 385 and hot or heated fluid to leave the pump unit 385.

Transiently holding cold or cooled fluid a reservoir space between “B” and “C”, allows the cold or cooled fluid to be heated by the heating elements 325 during the suction stroke and provides sufficient residence time in the pump cylinder 310 to heat the fluid to a desired temperature.

The methods and devices disclosed herein provide continuously heated fluid to the balloon 170 for the thermal treatments described above. If such a reservoir is not provided, and if the fluid that returns from the balloon catheter 160, is pumped immediately, without having residence time at or near a heating element, the fluid may not have sufficient residence time to be reheated prior to being recirculated into the balloon 170.

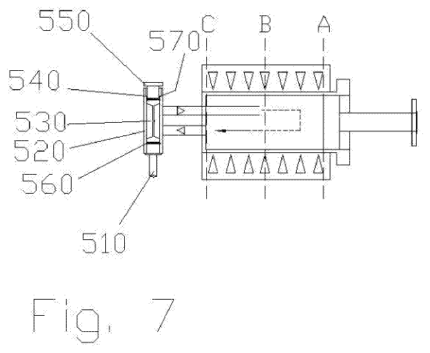

Turning to FIG. 6, a diagrammatic view of at least one embodiment of the programmable electronic controller system 350 is illustrated for exemplary purposes. The programmable electronic controller system 350 comprises a display 500 for displaying information about the operation and control of the vaginal remodeling device, e.g., temperature (temp) of the fluid, volume of fluid pumped as a percentage (cycle), rate the fluid is pumped (rate), the duration of the treatment (time set), elapsed time (time elapsed), and other operation and control parameters. The display may also display and control the pacing mode when pacing is used for treatment.

Turning to FIG. 7, a diagrammatic representation of one at least one embodiment of the feature that can prevent the reuse of the vaginal remodeling system 10 (see FIGS. 1-3). The pump unit may comprise a locking mechanism 505 to prevent reuse of the vaginal remodeling device. A connector 510 can be affixed to the housing 520 in the pump unit 385. There can be “0” rings made from a suitable material, like rubber, on both ends of the connector 510 to hermetically seal fluids in the pump unit 385 to prevent leakages during operation. The connector 510 may comprise a notch 540 for coupling the connector 510 to the housing 520 when it is fully inserted and prevent the connector 510 from decoupling from the housing 520. The connector 510 comprises at least one rib 560 to connect the proximal end 220 of the connector 510 to the distal end 550 of the housing 520 and has a notch at location 530. Once the connector 510 is introduced into the housing, it cannot be removed without destroying its function due to this locking mechanism 505. Once the operation is completed, the connector 510 can be removed by holding the connector in place proximally and turning the distal end 550. Due to this action, the connector 510 will snap at the notch location 530 and the connector 510 can be separated from the housing 520. The connector 510 is now dysfunctional and cannot be used again. By this means, the vaginal remodeling system 10 cannot be used again. This principal is universally applicable and connector devices can be designed in such manner to become ineffective when it is decouples from other connections in the medical procedure applications.

The illustrations in FIGS. 1-7 are meant to serve a general description of the concept of vaginal rejuvenation by balloon dilatation, providing un-localized thermal energy to the vaginal wall and optionally, using electrical pacing to create vaginal muscle contractions either all together or one or two at a time. The vaginal remodeling device and method of use improves cellular activity to promote collagen remodeling and vaginal competence. Alternate mechanisms and combinations thereof are possible and may be obvious to someone knowledgeable in the art based on this disclosure.

Although preferred embodiments of the invention have been described in detail herein, those skilled in the art will recognize that various substitutions and modifications may be made without departing from the scope and spirit of the appended claims.

1-18. (canceled)

19. A method for treating Vaginal Relaxation Syndrome (VRS) or Atrophic Vaginitis in a patient in need thereof, the method comprising the steps of:
a) inserting a dilation balloon into the patient's vagina; and
b) stretching and relaxing the vagina's vaginal wall with the dilation balloon, comprising:
i) mechanically inducing stress in the vaginal wall via inflating and deflating the dilation balloon, thereby stretching and relaxing the vaginal wall; and/or
ii) heating the vaginal wall by adding a heated fluid to the dilation balloon's interior, thereby uniformly heating the vaginal wall;
whereby mechanical energy and/or heat energy stimulate vaginal tissue cell function, activate collagen and elastin remodeling, and/or enhance blood flow to the vaginal wall, thereby treating the patient's VRS or Atrophic Vaginitis.

20. The method of claim 1, wherein an inflation and deflation cycle comprises a single inflation and deflation of the dilation balloon, and wherein step (b)(1) comprises one to ten inflation and deflation cycles per minute.

21. The method of claim 1, wherein the heated fluid of step (b)(ii) is water or saline and is pumped into the balloon by an inflation pump.

22. The method of claim 1, wherein the heated fluid of step (b)(ii) is heated by a low voltage heating element within the dilation balloon.

23. The method of claim 1, wherein step (a) is performed with the dilation balloon in a deflated state.

24. The method of claim 23, further wherein the dilation balloon is in a folded state.

25. The method of claim 24, wherein the dilation balloon comprises a low profile and is relatively non-distensible or semi-compliant, wherein the low profile comprises a configuration for easy insertion into the vagina, wherein relatively non-distensible comprises maintaining essentially the original dimensions, and wherein semi-compliant comprises being capable of growing in diameter by up to 5-10%.

26. The method of claim 19, wherein a flexible catheter is attached to the dilation balloon, wherein the catheter comprises a diameter ranging from 10 mm to 40 mm, and wherein the dilation balloon comprises a diameter ranging from 1 cm to 4 cm.

27. The method of claim 19, wherein the dilation balloon is inflated with an inflation syringe using water or saline.

28. The method of claim 19, wherein the dilation balloon is inflated with an inflation pump capable of providing a continuous stream of heated inflation fluid.

29. The method of claim 28, wherein the inflation pump is configured to control the inflation and deflation of the dilation balloon according to a preprogrammed cycle, thereby creating cycles of inflation and deflation of the dilation balloon while it is inserted in the patient's vagina.

30. The method of claim 29, wherein the inflation pump is also configured to heat and maintain the temperature of the inflation fluid.

31. The method of claim 19, wherein the dilation balloon further comprises one or more electrodes on the dilation balloon's surface and step (b) further comprises providing electrical stimulation to the vaginal wall to cause vaginal tube muscle contractions, whereby electrical stimulation stimulates vaginal tissue cell function, activates collagen and elastin remodeling, and/or enhances blood flow to the vaginal wall, thereby treating the patient's VRS or Atrophic Vaginitis.

32. The method of claim 31, wherein the electrical stimulation is pulsed.

33. A vaginal remodeling system comprising:
a) a dilation balloon configured to be inserted into a vagina of any size;
b) a flexible catheter attached to and in fluid communication with the dilation balloon;
c) pumping means configured to pump a fluid into and out of the dilation balloon via the catheter; and
d) heating means configured to maintain a desired temperature for the fluid.

34. The vaginal remodeling system of claim 33, wherein the catheter comprises a diameter ranging from 10 mm to 40 mm, and wherein the dilation balloon comprises a diameter ranging from 1 cm to 4 cm.

35. The vaginal remodeling system of claim 33, wherein the fluid comprises water or saline and the pumping means comprises an inflation syringe.

36. The vaginal remodeling system of claim 33, wherein the fluid comprises water or saline and the pumping means comprises an inflation pump.

37. The vaginal remodeling system of claim 36, further comprising a programmable electronic controller system for controlling the inflation pump, wherein the inflation pump is configured to control the inflation and deflation of the dilation balloon according to a preprogrammed cycle, thereby creating cycles of inflation and deflation of the dilation balloon while it is inserted in the patient's vagina.

38. The vaginal remodeling system of claim 37, wherein the inflation pump further comprises the heating means.

39. The vaginal remodeling system of claim 38, wherein the inflation pump is capable of providing a continuous stream of heated inflation fluid.

40. The vaginal remodeling system of claim 33, further comprising one or more electrodes on the dilation balloon's surface configured to provide electrical stimulation to the vaginal wall to cause vaginal tube muscle contractions and means for controlling the one or more electrodes, whereby electrical stimulation stimulates vaginal tissue cell function, activates collagen and elastin remodeling, and/or enhances blood flow to the vaginal wall, thereby treating the patient's VRS or Atrophic Vaginitis.

41. The vaginal remodeling system of claim 40, wherein the electrical stimulation is pulsed and/or variable.
